(19) United States
(12) Patent Application Publication
(10) Pub. No.: US 2018/0028072 A1
(43) Pub. Date: Feb. 1, 2018

(54) Wearable thermometer patch capable of measuring human skin temperature at high duty cycle

(71) Applicant: VivaLnk, Inc., Santa Clara, CA (US)

(72) Inventor: Wei Shi, San Jose, CA (US)

(21) Appl. No.: 15/590,657

(22) Filed: May 9, 2017

Related U.S. Application Data
(63) Continuation-in-part of application No. 15/406,380, filed on Jan. 13, 2017.
Application No. 15/406,380 is a continuation-in-part of application No. 15/224,121, filed on Jul. 29, 2016.

(51) Int. Cl. A61B 5/01; A61B 5/00

(52) U.S. Cl. CPC A61B 5/01; A61B 5/6833; A61B 5/0008; A61B 2560/0214

(57) ABSTRACT

A wearable thermometer patch for continuous wearing by a user includes a circuit substrate that includes an electric circuit, a battery holder mounted in the circuit substrate, and a detachable cover layer on the battery holder. The battery holder can hold a replaceable battery to supply power to the electric circuit. A temperature probe unit in connection with the electric circuit includes one or more temperature sensors in electric connection with the electric circuit in the circuit substrate. The one or more temperature sensors each can measure temperatures near the user's skin to produce one or more temperature values.

### BACKGROUND OF THE INVENTION

The present application relates to electronic devices, and in particular, to electronic patches that can attach to human skin for conducting measurement.

Electronic patches can be used for tracking objects and for performing functions such as producing sound, light or vibrations, and so on. As applications and human needs become more sophisticated and complex, electronic patches are required to perform a rapidly increasing number of tasks. Electronic patches are often required to be conformal to curved surfaces, which in the case of human body, can vary overtime.

Electronic patches can communicate with smart phones and other devices using WiFi, Bluetooth, Near Field Communication (NFC), and other wireless technologies. NFC is a wireless communication standard that enables two devices to quickly establish communication within a short range around radio frequency of 13.56 MHz. NFC is more secure than other wireless technologies such as Bluetooth and Wi-Fi because NFC requires two devices in close proximity (e.g. less than 10 cm). NFC can also lower cost comparing to other wireless technologies by allowing one of the two devices to be passive (a passive NFC tag).

Bluetooth is another wireless communication standard for exchanging data over longer distances (in tens of meters). It employs short wavelength UHF radio waves from 2.4 to 2.485 GHz from fixed or mobile devices. Bluetooth devices have evolved to meet the increasing demand for low-power solutions that is required for wearable electronics. Benefited from relatively longer reading distance and active communication, Bluetooth technologies allow wearable patches to continuously monitoring vital information without human interference, which is an advantage over NFC in many applications.

Wearable patch (or tag) is an electronic patch to be worn by a user. A wearable patch is required to stay on user's skin and operate for an extended period of time from hours to months. A wearable patch can contain a micro-electronic system that can be accessed using NFC, Bluetooth, WiFi, or other wireless technologies. A wearable patch can be integrated with different sensors such as vital signs monitoring, motion track, skin temperature measurements, and ECG detection.

Despite recent development efforts, current wearable patches still suffer several drawbacks: they may not provide adequate comfort for users to wear them; they may not stay attached to user's body for the required length of time; and they are usually not aesthetically appealing. The conventional wearable patches also include rigid polymer substrates that are not very breathable. The build-up of sweat and moisture can cause discomfort and irritation to the skin, especially after wearing it for an extended period of time.

Conventional wearable thermometer patches have the additional challenge of inaccurate temperature measurement due to factors such as thermal resistance between the temperature sensor and the human skin, conduction loss of the temperature sensor to the ambient environment, as well as temperature reduction in the user skin caused by the thermal conduction to the wearable patch. Moreover, conventional wearable thermometer patches can also have slow measurement responses.

Another challenge for conventional wearable thermometer patches is that the user's skin may interfere with their proper wireless communications. For example, the antenna's communication range can be significantly reduced by the adjacency to user's skin. The wireless communication range of an antenna in contact with the skin is less than half the range for an antenna that is placed 4 mm away from the user's skin.

Yet another challenge is that it is extremely difficult to measure the surface temperature accurately, especially when measuring the human skin temperature which being impacted by the blood circulation under the skin. Several critical factors can impact the continuous measurement of armpit temperature: the ambient temperature can impact temperature measurement when arm is opened; and thermal contact resistance can change when the contact between the temperature probe and human skin became loose.

Still another challenge is that conventional wearable patches are usually powered by rechargeable batteries that typically last a couple of days and require charging for a couple of hours in between usages. The duty cycles of these conventional wearable patches are not quite compatible with continuous monitoring human bio-signals.

There is therefore a need for a flexible wearable electronic patch that can correctly measure temperatures of user's skin at high accuracy, fast response time, and high duty cycle, while capable of performing wireless communications in a required range.

### SUMMARY OF THE INVENTION

The presently disclosure attempts to address the aforementioned limitations in conventional electronic patches. The presently disclosed wearable wireless thermometer patch that can be attached to human skin to conduct temperature measurements with high accuracy and faster respond time.

In the presently disclosed wearable wireless thermometer patch, temperature measurement errors due to the thermal noise from the environment are minimized. In metrology, accurate metrology instrument is associated with high Signal-to-Noise Ratio (SNR). In the presently disclosed wearable thermometer patch, the thermal resistance between the temperature sensor and the human skin is minimized, so that the maximum amount of heat can be conducted quickly from the user skin to the temperature sensor. Moreover, the heat conduction loss from the temperature sensor to the ambient is also minimized by the structure design and thermal material. Furthermore, a perforated protective film is placed between the user skin and the body of the wearable patch to reduce the heat conduction from the user skin, because the conventional non-perforated film will lower down the true temperature of the skin due to the attachment of the wearable patch. In addition, the presently disclosed wearable thermometer patch is structured to have low thermal capacity which results in faster responding time as well as higher flexibility.

Furthermore, the disclosed electronic patches are also breathable and stretchable. The stretchability and the breathability make the disclosed electronic patches more comfortable for the users. The disclosed electronic patches are capable wireless communication with little interference from users' skins. Moreover, the disclosed electronic patches can conduct measurements both at users' skins and away from the user's skin. The present application further discloses simple and effective manufacturing process to fabricate such wearable electronic patches.

Additionally, the disclosure teaches a wearable wireless thermometer patch structure that can be attached to human skin for the correct temperature measurement with the double temperature sensors (DTS) and a force sensor. Using DTS, the temperature under the dermis can be easily calculated from the Fourier's Law at the thermal equilibrium status, which is independent of the ambient temperature changes when the arm is open or closed. By integrating the force sensor, the thermal contact resistance can be easily correlated to the contacting force, from which the armpit temperature can be calculated more accurately regardless the arm is lightly or tightly in contact with the thermometer patch.

Moreover, the disclosed wearable patches can include battery holders compatible with easily replaceable batteries, which enable high measurement duty cycle and continuous measurements of human skin temperature and other bio vital signals.

Another advantageous feature of the disclosed wearable patches is that the easily removed batteries allow shipments of the disclosed wearable patches without batteries, which can improve shipment safety of the wearable patches, as regulations have become stricter to the transportation of batteries.

In one general aspect, the present invention relates to a wearable thermometer patch for continuous wearing by a user, which includes a circuit substrate comprising an electric circuit; a battery holder mounted in the circuit substrate, wherein the battery holder can hold a replaceable battery to supply power to the electric circuit; a temperature probe unit in connection with the electric circuit, comprising one or more temperature sensors in electric connection with the electric circuit in the circuit substrate, in which the one or more temperature sensors each can measure temperatures near the user's skin to produce one or more temperature values; and a detachable cover layer on the battery holder.

Implementations of the system may include one or more of the following. The wearable thermometer patch can further include a stretchable and permeable layer below the circuit substrate and the battery holder, wherein the temperature probe unit is mounted in an opening of the stretchable and permeable layer, wherein at least a portion of the temperature probe unit can be in contact with the user's skin. The temperature probe unit can include a thermally conductive cup having a bottom portion configured to be in contact with the user's skin, wherein the one or more temperature sensors are placed inside the thermally conductive cup and are in thermal conduction with the thermally conductive cup. The wearable thermometer patch can further include one or more spacers on the stretchable and permeable layer; and a thin film on the one or more spacers, wherein the detachable cover can be adhesively attached to a portion of the thin film. The one or more spacers can include a wedge-shaped spacer that defines varying thicknesses for the wearable thermometer patch. The wedge-shaped spacer can include a thinner side and a thicker side, wherein the thicker side is adjacent to the circuit substrate and the battery holder. The wearable thermometer patch can further include an elastic cover layer between the detachable cover layer and the battery holder holding the associated replaceable battery therein. The temperature probe unit can include a housing; a first plate in the housing; and a first pair of temperature sensors in the housing, including: a first temperature sensor attached to a lower surface of the first plate; and a second temperature sensor under the first temperature sensor and attached to an upper surface of the first plate. The wearable thermometer patch can further include a thermally insulating material in the housing, which encapsulates the first pair of temperature sensors. The wearable thermometer patch can further include a second pair of temperature sensors in the housing, including: a third temperature sensor attached to a lower surface of the first plate; and a fourth temperature sensor under the third temperature sensor and attached to an upper surface of the first plate, wherein the first plate has a first thickness between the first pair of temperature sensors and a second thickness between the second pair of temperature sensors. The semiconductor chip can calculate the temperature of the user's skin in part using a difference between temperature values respectively measured by the third temperature sensor and the fourth temperature sensor. The first thickness can be different from the second thickness. The wearable thermometer patch can further include a second plate separated from the first plate by a gap in a planar direction; a second pair of temperature sensors in the housing, including: a third temperature sensor attached to a lower surface of a second plate; and a fourth temperature sensor under the third temperature sensor and attached to an upper surface of the second plate, wherein the first plate can have a first thickness between the first pair of temperature sensors, wherein the second plate can have a second thickness between the second pair of temperature sensors. The semiconductor chip can calculate the temperature of the user's skin in part using a difference between temperature values respectively measured by the third temperature sensor and the fourth temperature sensor. The first thickness can be different from the second thickness. The first thickness can be substantially the same as the second thickness. The wearable thermometer patch can further include a semiconductor chip mounted on the circuit substrate and in electric connection with the electric circuit, wherein the semiconductor chip can receive electric signals from the one or more temperature sensors in response to respective temperatures measured from the user's skin. The semiconductor chip can calculate the temperature of the user's skin in part using a difference between temperature values respectively measured by the first temperature sensor and the second temperature sensor. The wearable thermometer patch can further include a thermal conductive spreader layer attached below the housing of the temperature probe unit and the circuit substrate. The wearable thermometer patch can further include a semiconductor chip mounted on the circuit substrate and in electric connection with the electric circuit; and an antenna in electric connection with the semiconductor chip, wherein the antenna can wirelessly send temperatures measured by the one or more temperature sensors or calculated temperature values to an external device.

In a general aspect, the present invention relates to a wearable thermometer patch that includes a substrate and a temperature probe unit mounted in the substrate and configured to measure temperature of a user's skin. The temperature probe unit can include a force sensor configured to measure contact force between the temperature probe unit and the user' skin, a plate, a first temperature sensor attached to a lower surface of the plate, and a second temperature sensor attached to an upper surface of the plate.

Implementations of the system may include one or more of the following. The substrate can include an electric circuit that is electrically connected to the first temperature sensor, the second temperature sensor, and the force sensor. The first temperature sensor and the second temperature sensor can be respectively configured to measure a first time series of temperature values and a second time series of temperature values, wherein the temperature of the user's skin is calculated by discarding at least a portion of the temperature values in the first time series of temperature values and the second time series of temperature values based on the contact force measured by the force sensor. The substrate can include an opening, wherein the temperature probe unit comprises a thermally conductive cup having a bottom portion mounted in the opening of the substrate. The wearable thermometer patch can further include a thermally-conductive adhesive that fixes the first temperature sensor, the second temperature sensor, and the plate to an inner surface of the thermally conductive cup. The wearable thermometer patch can further include a thermally insulating material in a top portion of the thermally conductive cup, wherein the force sensor is positioned on the thermally insulating material and the thermally conductive cup. The wearable thermometer patch can further include a controller mounted on the flexible circuit substrate and in electric connection with the electric circuit, wherein the controller can receive first electric signals from the first temperature sensor and the second temperature sensor in response to respective temperature measurements, wherein the controller can receive second electric signals from the force sensor in response to measurement of the contact force. The controller can calculate the temperature of the user's skin using a difference between temperature measurements from the first temperature sensor and the second temperature sensor. The controller can segment a time series of the temperature measurements from the first temperature sensor and the second temperature sensor based on the second electric signals received from the force sensor. The controller can calculate the temperature of the user's skin by discarding at least a portion of the temperature values in the first time series of temperature values and the second time series of temperature values based on the contact force measured by the force sensor. The wearable thermometer patch can further include an antenna in electric connection with the semiconductor chip, wherein the antenna to wirelessly send measured temperature values and contact force values to an external device. The wearable thermometer patch can further include electronic components mounted or formed on the flexible circuit substrate and in electric connection with electric circuit, wherein the electronic components can include a semiconductor chip, an antenna, a battery, or a bonding pad. The wearable thermometer patch can further include an elastic layer formed on the substrate and the temperature probe unit. The wearable thermometer patch can further include an adhesive layer under the substrate, the adhesive layer configured to attach to human skin.

These and other aspects, their implementations and other features are described in detail in the drawings, the description and the claims.

### BRIEF DESCRIPTION OF THE DRAWINGS

FIG. 1 illustrates the usage of a wearable patch attached to a user's skin.

FIG. 2 is a cross-sectional view of a base structure for constructing a wearable thermometer patch in accordance with some embodiments of the present invention.

FIG. 3 is a cross-sectional view of a wearable thermometer patch capable of conducting accurate and fast-response temperature measurements and effective wireless communications in accordance with some embodiments of the present invention.

FIG. 4 is a detailed cross-sectional view of the temperature sensing portion in the wearable thermometer patch in FIG. 3.

FIG. 5 is a cross-sectional view of an improved wearable thermometer patch including a DTS and a force sensor to assist correct temperature measurements in accordance with some embodiments of the present invention.

FIG. 6 is a detailed cross-sectional view of the temperature sensing portion in the wearable thermometer patch shown in FIG. 5.

FIG. 7 illustrates time series of temperature and force measurement data and segmentation of the temperature measurement data based on the force measurement data.

FIG. 8 is a cross-sectional view of a wearable thermometer patch capable of conducting accurate and fast-response temperature measurements at high duty cycle and effective wireless communications in accordance with some embodiments of the present invention.

FIG. 9 is a cross-sectional view of another wearable thermometer patch capable of conducting accurate and fast-response temperature measurements at high duty cycle and effective wireless communications in accordance with some embodiments of the present invention.

FIG. 10 is a cross-sectional view of another wearable thermometer patch capable of conducting accurate and fast-response temperature measurements at high duty cycle and effective wireless communications in accordance with some embodiments of the present invention.

FIGS. 11A-11C are detailed cross-sectional views of different implementations of the temperature probe unit in the wearable thermometer patch in FIG. 10.

### DETAILED DESCRIPTION OF THE INVENTION

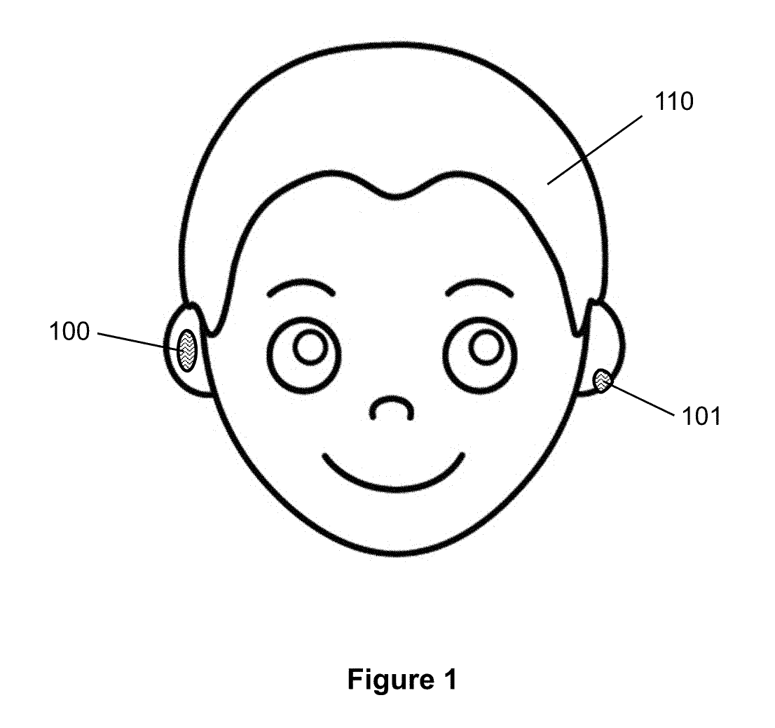

Referring to FIG. 1, one or more dual purpose wearable patches 100, 101 are attached to the skin of a user 110 for measuring body vital signs. The dual purpose wearable patch 100 can be placed on the ears, the forehead, the hands, the shoulder, the waist, the leg, or the foot, under the armpit, around the wrist, on or around the arm, or other parts of a user's body. In the present disclosure, the term “wearable patch” can also be referred to as “wearable sticker”, “wearable tag”, or “wearable band”, etc.

As discussed in more detail below, dual purpose wearable patches 100, 101 can operate individually, or in a group to provide certain desired treatment or measurement. For example, the purpose wearable patch 101 can wrap around a user's ear for applying an electric field through certain location of the ear. Similar, the disclosed purpose wearable patch can wrap around a user's wrist for providing treatment and measurement. Moreover, the dual purpose wearable patches 100, 101 can be attached to different parts of a user's body such as on the two ears or the two temples of the user 110, which allows a low electric voltage signal to be applied across the user's head.

As discussed above, wearable electronic patches face several challenges: the user's skin may interfere with their proper operations. For example, the wearable patch 100 may include an antenna for wireless communications with other devices. The antenna's communication range can be significantly reduced when an antenna is placed in contact with the user's skin.

The presently disclosure aims to overcome the drawbacks in conventional wearable patches, and to provide highly stretchable, compliant, durable, breathable, and comfortable wearable electronic patches while performing more accurate and more responsive measurements and communication functions.

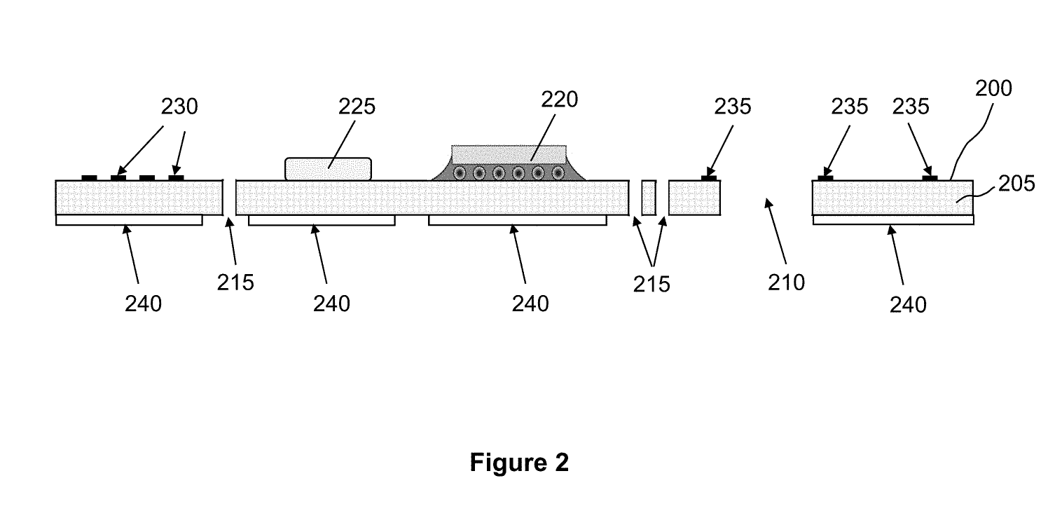

Referring to FIG. 2, a base structure 200 includes a flexible circuit substrate 205 having an electric circuit embedded in or formed on. The flexible circuit substrate 205 has a large opening 210 and multiple small through holes 215. A semiconductor chip 220, a battery 225, an antenna 230, and bonding pads 235 are mounted or formed on the upper surface of the flexible circuit substrate 205. The semiconductor chip 220, the battery 225, the antenna 230, and at least one of the bonding pads 235 is connected with the electric circuit in the flexible circuit substrate 205.

Stiffening layers 240 are formed on the layer surface of the flexible circuit substrate 205 at locations respectively below electronic components such as the semiconductor chip 220, the battery 225, the antenna 230, and the bonding pads 235. The stiffening layers 240 have higher Young's modulus than that of the flexible circuit substrate 205, and can protect the electronic devices from being damaged when the flexible circuit substrate 205 is bent. The flexible circuit substrate 205 can be made of polymeric materials and built in with electric circuitry that connects the semiconductor chip 220, the battery 225, the antenna 230, and the bonding pads 235. The stiffening layers 240 can be made of metallic or polymeric materials.

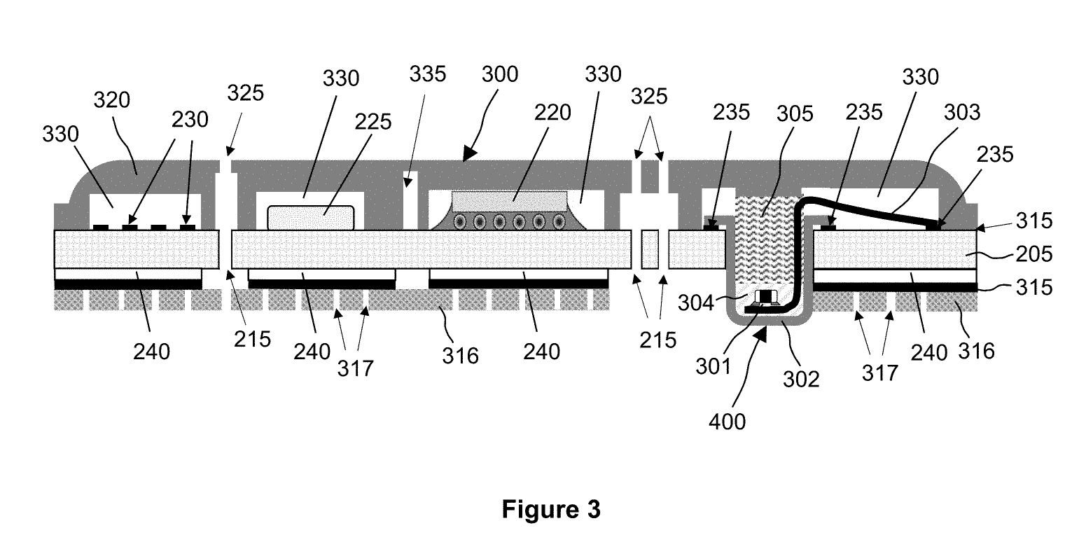

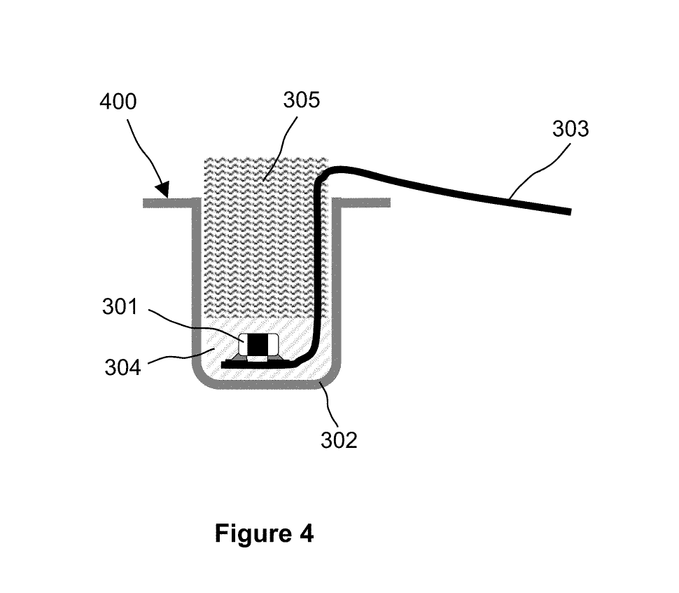

Referring to FIGS. 3 and 4, a wearable thermometer patch 300 includes a temperature probe unit 400, in addition to the components in the base structure 200 as shown in FIG. 2. In the temperature probe unit 400, a thermally conductive cup 302 has its bottom portion plugged into the large opening 210 (FIG. 2). The bottom portion of the thermally conductive cup 302 protrudes out of the lower surface of the flexible circuit substrate 205. The lips of the thermally conductive cup 302 near its top portion are fixedly attached or bonded to bonding pads 235 by soldering or with an adhesive. The thermally conductive cup 302 can be made of a thermally conductive metallic or alloy material such as copper, stainless steel, ceramic or carbide composite materials. A temperature sensor 301 is attached to and in thermal conduction with an inner surface near the bottom of the thermally conductive cup 302. The temperature sensor 301 can be implemented, for example, by a Thermistor, a Resistor Temperature Detector, or a Thermocouple. When an outer surface of the bottom portion of the thermally conductive cup 302 is in contact with a user's skin, the thermally conductive cup 302 can thus effectively transfer heat from a user's skin to the temperature sensor 301. A flexible conductive ribbon 303 is connected to the temperature sensor 301 in the thermally conductive cup 302 and one of the conductive pads 235 on the flexible circuit substrate 205. Thus the temperature sensor 301 is connected to the electric circuit in the flexible circuit substrate 205 and can send an electric signal to the electric circuit and the semiconductor chip 220 in response to temperature measured by the temperature sensor 301. The semiconductor chip 220 processes the electric signal and outputs another electrical signal which enables the antenna 230 to transmit a wireless signal to send measurement data to another external device such as a mobile phone or a computer. The battery 225 powers the semiconductor chip 220, the electric circuit, and possibly the temperature sensor 301.

The temperature sensor 301 and a portion of the flexible conductive ribbon 303 are fixed to an inner surface at the bottom of the thermally conductive cup 302 by a thermally-conductive adhesive 304, which allows effective heat transfer from the bottom of the thermally conductive cup 302 to the temperature sensor 301. Examples of the thermally-conductive adhesive 304 can include electrically-insulative thermally-conductive epoxies and polymers. A thermally insulating material 305 is fixed in and fills the top portion of the thermally conductive cup 302, which fixes the thermally-conductive adhesive 304 at the bottom of the thermally conductive cup 302 and reduces heat loss from the temperature sensor 301 to the elastic layer (described below) or the environment. The flexible conductive ribbon 303 can be bent and laid out along the wall the thermally conductive cup 302.

A layer of a perforated polymer material 316 is bonded to the bottom surface of the flexible circuit substrate 205 using adhesive material 315. Suitable material for the perforated polymer material 316 can include soft materials such as Polyurethane. The layer of perforated polymer material 316 can include multiple holes 317: one of them exposes a bottom of the thermally conductive cup; others allow sweat and moisture to escape through holes 215 and holes 325; while other holes 317 help enhance flexibility and comfort of the perforated polymer material. An adhesive material is applied to the lower surface of the perforated polymer material 316 to be attached the lower surface of the perforated polymer material 316 to the user's skin, so that the bottom of the thermally conductive cup 302 can be in tight contact with a user's skin for the accurate temperature measurement of the user's skin.

It should be noted that when the wearable thermometer patch 300 is worn by the user, the antenna 230 is separated from the user's skin by the flexible circuit substrate 205 and the layer of the perforated polymer material 316, which minimizes the impact of the user's body on the transmissions of wireless signals by the antenna 230.

An elastic layer 320 is bonded onto the upper surface of the flexible circuit substrate 205 with an adhesive material 315 in between. Alternatively, the elastic layer 320 can directly be molded onto the flexible circuit substrate 205 without using any bonding interface material 315. The elastic layer 320 includes recesses 330 on the underside to define cavities to contain the antenna 230, the battery 225, the semiconductor chip 220 and the flexible conductive ribbon 303. The elastic layer 320 also includes holes 325 that are registered to the through holes 215 in the flexible circuit substrate 205, which allows moisture and sweat from the user's skin to diffuse to the ambient environment, which enhances user's comfort and strength of attachment of the wearable thermometer patch 300 to the user's skin. The elastic layer 320 can include one or more cavities 335 for enhancing flexibility (bendable) and stretchability of the elastic layer 320 and the whole wearable thermometer patch 300. The cavities 335 can have elongated shapes with lengthwise direction oriented perpendicular to the flexible circuit substrate 205.

The elastic layer 320 can be made of a non-conductive material such as an elastomeric material or a viscoelastic polymeric material having low Young's modulus and high failure strain. In some embodiments, the elastic layer 320 has a Young's Modulus<0.3 Gpa. In some cases, the elastic layer 320 and can have Young's Modulus<0.1 Gpa to provide enhanced flexibility and tackability. Materials suitable for the elastic layer 320 include elastomers, viscoelastic polymers, such as silicone, silicone rubber, and medical grade polyurethane that is a transparent medical dressing used to cover and protect wounds with breathability and conformation to skin.

The disclosed wearable thermometer patch can significantly enhance measurement accuracy and responsiveness, and reduce thermal noise. The temperature sensor is positioned very close to a user's skin. The temperature sensor is placed at the bottom of a thermally conductive cup and in good thermal conduction with the user's skin. The minimized thermal resistance between the temperature sensor and the user's skin reduces temperature measurement error and also decreases measurement response time. Moreover, the temperature sensor is secured fixed by an adhesive to the bottom of the thermally conductive cup such that the temperature sensor is not affected and detached by user's body movements, which improves durability of the wearable thermometer patch. Furthermore, the temperature sensor is thermally isolated with the ambient environment by a thermal insulating material in the top portion of the thermally conductive cup. The reduced thermal capacity helps further reduces background noise in the measurements of user's skin temperature and increase response rate of measurement. A layer of soft perforated polymer material under the flexible substrate minimizes heat conduction from the user's skin to the wearable thermometer patch, thus reducing the “cooling effect” of the user's skin by the wearable thermometer patch.

Another advantage of the disclosed wearable thermometer patch is that it is stretchable, compliant, durable, and comfortable to wear by users. The disclosed wearable thermometer patch includes a flexible substrate covered and protected by an elastic layer that increases the flexibility and stretchability. Cavities within the elastic layer further increase its flexibility and stretchability. A layer of soft perforated polymer material under the flexible substrate provides comfortable contact to user's skin is in contact with user's skin. Openings in the elastic layer, the substrate, and the soft perforated polymer material can bring moisture and sweat from the user's skin to the ambient environment, which increases user's comfort as well as strength of the attachment of the wearable thermometer patch to user's skin.

Yet another advantage of the disclosed wearable thermometer patch is that it can significantly increase wireless communication range by placing the antenna on the upper surface of the flexible circuit substrate. The thickness of the substrate as well as the height of the thermally conductive cup can be selected to allow enough distance between the antenna and the user's skin to minimize interference of user's body to the wireless transmission signals.

Further details of wearable thermometer patches are disclosed in the commonly assigned co-pending U.S. patent application Ser. No. 14/814,347 “Three dimensional electronic patch”, filed Jul. 30, 2015, the disclosure of which is incorporated herein by reference.

In some embodiments, the present disclosure teaches an improved thermometer patch that can properly compensate for the status of the physical contacts (no contact, loose contact, or tight contact, etc.) between the thermometer patch and the user's body.

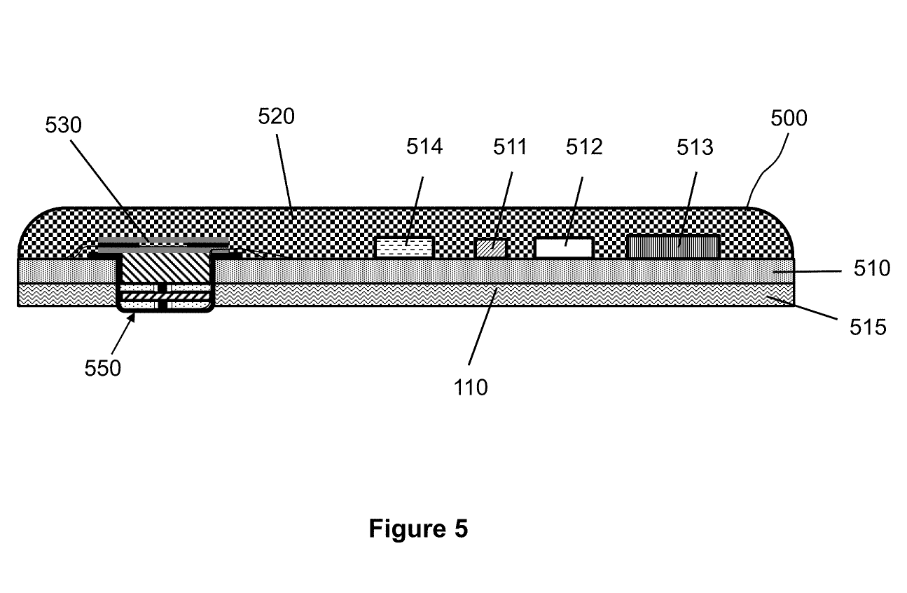

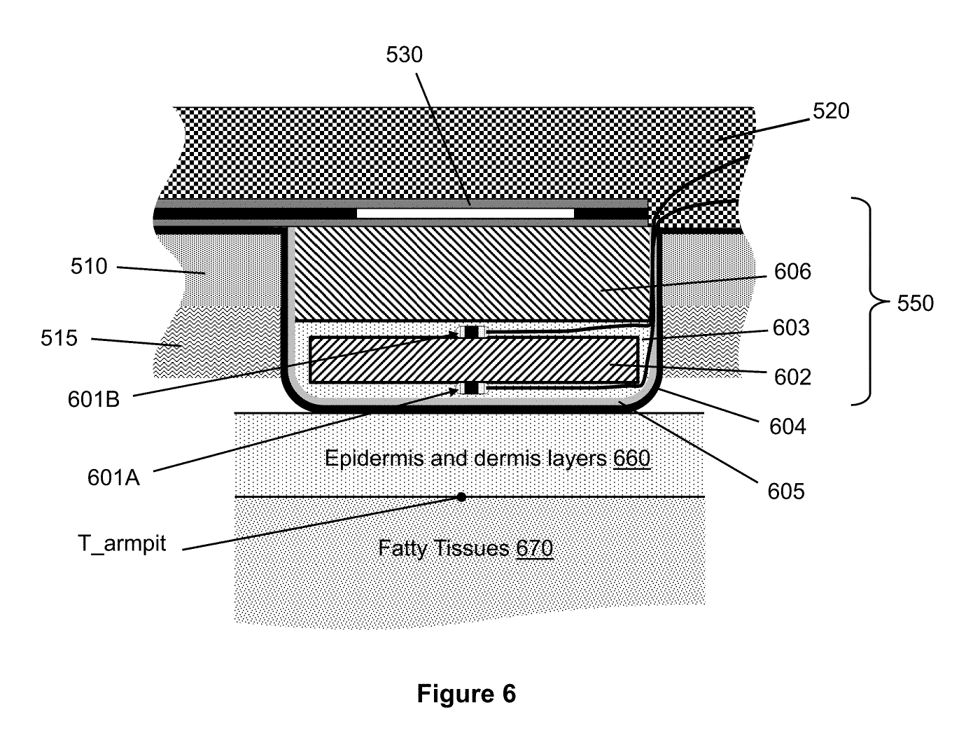

Referring to FIGS. 5 and 6, an improved wearable thermometer patch 500 includes a temperature probe unit 550, a substrate 510, and a RF antenna 511, a Bluetooth chip 512, a battery 513, and a controller 514 mounted on the substrate 510. An adhesive layer formed under the substrate 510 can attach the improved wearable thermometer patch 500 to human skin. The substrate 510 can be implemented by a flexible printed circuit board (PCB), a printed PET, or a PCB). The RF antenna 511, the Bluetooth chip 512, the battery 513, and the controller 514 are electrically connected a circuit (not shown) in the substrate 510. An elastic layer 520 is formed on the temperature probe unit 550, the substrate 510, the RF antenna 511, the Bluetooth chip 512 and the battery 513. The elastic layer 520 can be formed by materials such as silicone, polyurethane, thermoplastic polyurethane, a polyethylene foam, or a fabric.

The temperature probe unit 550 includes temperature sensors 601A and 601B which are respectively bonded to the bottom surface and the top surface of a plate 602. The plate 602 has a known thermal resistance, which can be formed by materials such as plastic, ceramic, metal, or foam materials. The temperature sensors 601A and 601B can be implemented for example by thermistor, a resistance temperature detector, or thermocouple, which are electrically connected to the circuit in the substrate 510. The temperature probe unit 550 also includes a metal cup 604 which is mounted in an opening in the substrate 510. The metal cup 604 can be formed with copper, stainless, ceramic, carbide, or other metallic alloys. An electrically insulating layer 605 is formed on an inner surface of the metal cup 604. The assembly of temperature sensors 601A and 601B and the plate 602 are attached to the metal cup 604 and the electrically insulating layer 605 therein by thermally-conductive epoxy 603. A thermally insulating material 606 fills up the metal cup 604 over the thermally-conductive epoxy 603.

The temperature probe unit 550 also includes a force sensor 530 attached to the top of the metal cup 604 and the thermally-insulating material 606 therein. The force sensor 530 is electrically connected to the circuit in the substrate 510, and can be implemented by a force sensitive resistor (FSR), a micromechanical electro (MEMS) strain sensor, or other types of force or pressure sensors. The elastic layer 520 is compressible when an external force is applied to the top of the improved wearable thermometer patch 500, which transmits a force to the force sensor 530.

When the improved wearable thermometer patch 500 is attached to a user's skin under the armpit, it is desirable to accurately measure the user's body temperature under the skin, at the interface between epidermis and dermis layers 660 and a fatty tissue layer 670.

In accordance with the present invention, the assembly of temperature sensors 601A and 601B and the force sensor 530 allows accurate measurement of the user's skin temperature. When the diameter of a plate is large enough, the temperature distribution across the surfaces is approximately uniform; one-dimensional Fourier's law can be applied to describe heat conduction in the thickness direction of the plate 602:

q=K(T1−T2)/Δx eqn.(1)

where q is the heat flux conducted through the plate; K is the thermal conductivity of the plate 602; T1 and T2 are respectively the temperatures measured by the temperature sensors 601A and 601B at the bottom and the top surfaces of the plate 602, while Δx is the thickness of the plate 602.

The epidermis and dermis layers 660, the bottom layer of the metal cup 604, the electrically insulating layer 605, and the layer of thermally-conductive epoxy 603 between the temperature sensor 601A and the electrically insulating layer 605 can also be modeled by a stack of plates. At thermal equilibrium, the heat flux conducted is the same through all the plates in the stack. The skin temperature under the epidermis and dermis layers 660 can be calculated based on one-dimensional Fourier's law with the following equation:

T_armpit=qΔx′/K′+T1 eqn.(2)

where T_armpit (shown in FIG. 6) is the skin temperature under the epidermis and dermis layers 660; K′ is the composite thermal conductivity of the above described layers, T1 is the temperature measured by the temperature sensor 601A at the bottom and the top surfaces of the plate 602, while Δx′ is the total thickness of these layers.

Equations (1) and (2) show that when the pair of the temperature sensors 601A and 601B are used to measure temperature across the plate 602, the measurement value of T_armpit is minimally impacted by the thermal environment above the elastic layer 520. In other words, when arm is opened, the heat convection in the air has little influence on the measurement of T_armpit.

The calculations described in equations (1) and (2) above can be conducted by the controller 514 or an external device wirelessly connected with the improved wearable thermometer patch 500 via the Bluetooth chip 512. The controller 514 can receive temperature measurement data from the temperature sensors 601A and 601B via the circuit in the substrate 510.

When arm is opened or closed, however, the thermal contact resistance between the bottom of the metal cup 604 and the epidermis and dermis layers 660 may vary. The integrated force sensor 530 can measure the contact force, which correlates with the thermal contact resistance. Thus, using a combination of DTS and a force sensor, a more accurate temperature can be obtained from the armpit by eliminating impacts from the ambient temperature and the compressing force and the variations in the contact force.

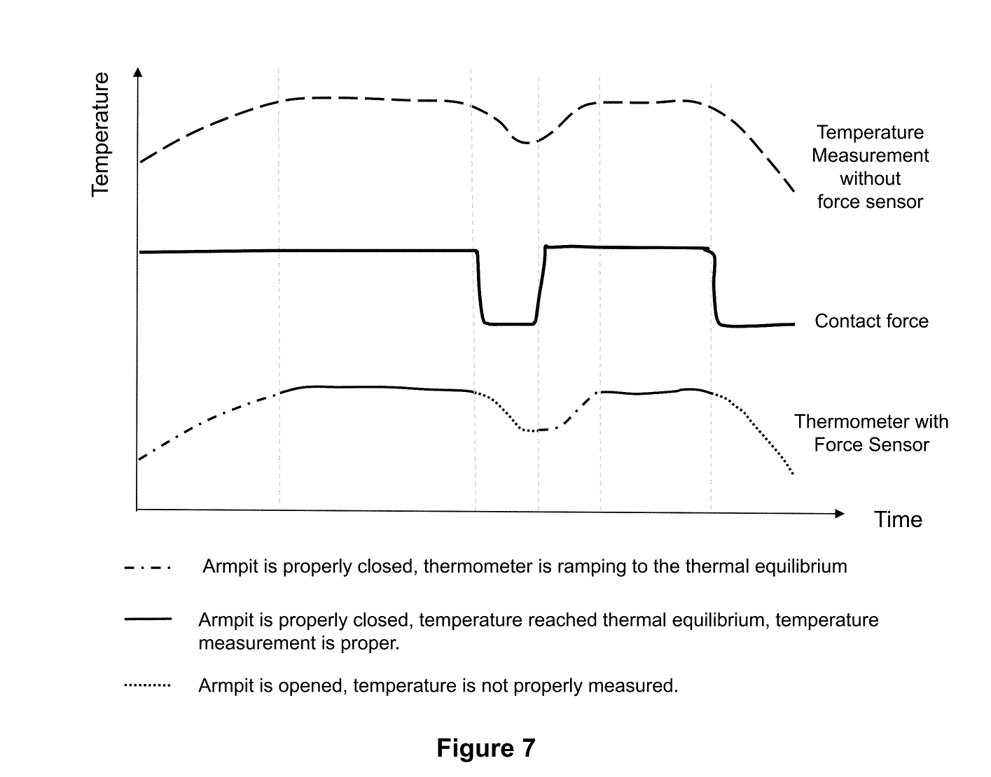

Referring to FIG. 7, the upper curve shows a time series of temperatures measured without contact force measurement, which shows unknown variations in temperature values, which are sources of measurement inaccuracies. The curve in the middle shows a time series of contact forces measured by the above described force sensor, which shows variations in the contact force, which is caused by the open and close of the armpit during measurements. The lower curve shows a time series of temperature measurement being segmented according to the open/close status of the armpit as interpreted by the contact force measurement by the force sensor: a) the dotted-dashed lines show the status when the armpit is properly closed and thermometer is ramping to the thermal equilibrium; b) the solid lines show that armpit is properly closed, the temperature have reached thermal equilibrium, and the temperature measurements are proper; and c) the dotted lines correspond to the period when the armpit is opened, temperature is not properly measured, and the temperature measurement data should be discarded. The temperature measurement of user's skin can thus be drastically improved by using data obtained from only the periods when there are good thermal contacts between the improved wearable thermometer patch 500 and the user's skin.

The above described segmentation and selection of the time series of the temperature measurement data based on force sensing data can be conducted by the controller 514 or an external device wirelessly connected with the improved wearable thermometer patch 500 via the Bluetooth chip 512. The controller 514 can receive temperature measurement data from the force sensor 530 via the circuit in the substrate 510.

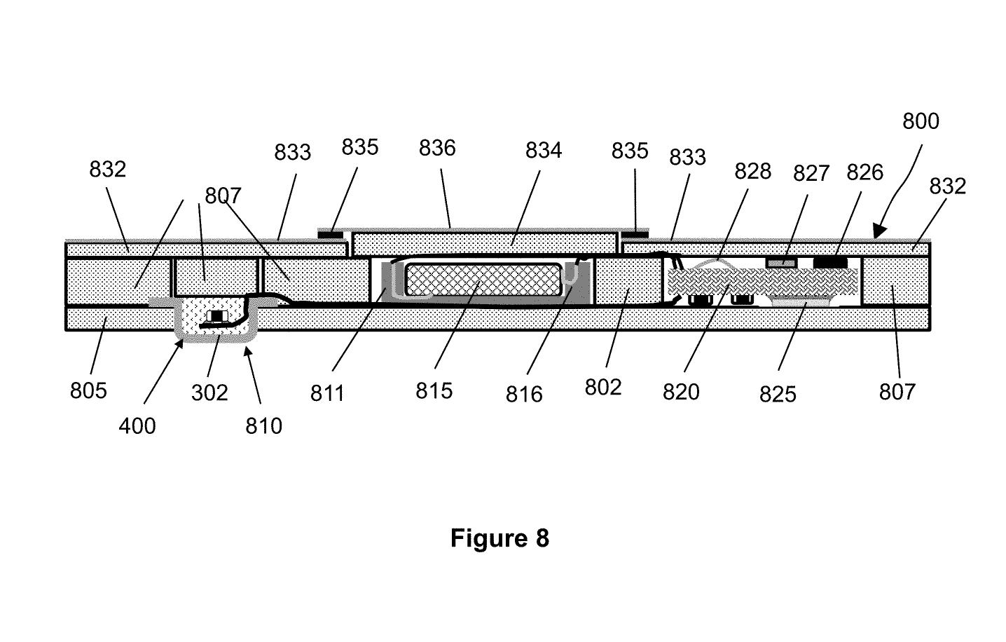

In some embodiments, referring to FIG. 8, a wearable thermometer patch 800 includes a stretchable and permeable layer 805 that include openings 810. The stretchable and permeable layer 805 can be made of soft foam material such as EVA, PE, CR, PORON, EPD, SCF or fabric textile, to provide stretchability and breathability. A temperature probe unit 400, with details described above and shown in FIG. 4, is mounted in the opening 810. A battery holder 811 is attached to an upper surface of the stretchable and permeable layer 805, in which a replaceable battery 815 can be mounted and electrically connected to connectors 816 to power electronic component such as the temperature probe unit 400. One or more spacers 807 having height similar or slightly higher than the battery holder 811 are also attached to the upper surface of the stretchable and permeable layer 805 to protect the battery holder 811 and a circuit substrate 820. The one or more spacers 807 can be formed by a soft foam material similar to that of the stretchable and permeable layer 805. A semiconductor chip 825, an antenna 826, an LED indicator 827, and a switch 828 are mounted on or under the circuit substrate 820. The circuit substrate 820 includes an electric circuit therein that electrically connects the various electronic components thereon to the connectors 816 and the replaceable battery 815. The LED indicator 827 can indicate the mode and status (e.g. in measurement mode, off mode, fever warning, etc.) of the wearable thermometer patch 800. The switch 828 is optional and can turn the power from the replaceable battery 815 on or off. In one implementation, the circuit substrate 820 can be implemented with a printed circuit board. The circuit substrate 820 mounted with the various electronic components is bonded to the stretchable and permeable layer 805 by an adhesive layer.

Referring to FIGS. 4 and 8, the temperature probe unit 400, as described above, includes a thermally conductive cup 302 having its bottom portion mounted into the large opening 810 and fixed to the stretchable and permeable layer 805 by an adhesive. A temperature sensor 301 is electrically connected to the electric circuit in the circuit layer 805 by a flexible conductive ribbon 303. The temperature sensor 301 is connected to the electric circuits in the circuit substrate 820 and can send an electric signal to the electric circuit and the semiconductor chip 825 in response to temperature values measured by the temperature sensor 301. The semiconductor chip 825 processes the electric signal and outputs another electrical signal which enables the antenna 826 to transmit a wireless signal to send measurement data to an external device such as a mobile phone or a computer. The replaceable battery 815 powers the semiconductor chip 825, the electric circuit, and possibly the temperature sensor 301.

Referring back to FIG. 8, an elastic layer 832 is formed on the one or more spacers 807, and the circuit substrate 820 mounted with various electronic components. A thin film 833 is formed on the elastic layer 450 with an adhesive for protection and cosmetic purposes. In an opening of the elastic layer 832, an elastic cover layer 834 is placed over the battery holder 811 and the replaceable battery 815, and on portions of the one or more spacers 807. The elastic cover layer 834 can be removed to allow removal or replacement of the replaceable battery 815. The elastic cover layer 834 is sealed and held in place by a detachable cover layer 836 with a ring-shaped detachable water resistant adhesive 835.

The elastic layer 832 and the elastic cover layer 834 can be formed by soft stretchable foam and permeable materials such as EVA, PE, CR, PORON, EPD, SCF, or fabric textile. Thus the circuit substrate 820 mounted with the various electronic components, and the replaceable battery 815 are sandwiched between and protected by the stretchable and permeable layer 805, and the elastic layer 832 and the elastic cover layer 834. The spacers 807 provide extra cushion and protection to the temperature probe unit 400 and the above mentioned components.

The semiconductor chip 825 processes the electric signal and outputs an electrical signal which enables the antenna 826 to transmit a wireless signal carrying the measurement data to another external device such as a mobile phone or a computer. The wireless signal can be based on using WiFi, Bluetooth, Near Field Communication (NFC), and other wireless standards. When the wearable thermometer patch 800 is worn by a user, the antenna 826 is separated from the user's skin by the thickness of the circuit substrate 416 and the stretchable and permeable layer 805, which minimizes the impact of the user's body on the transmissions of wireless signals by the antenna 826.

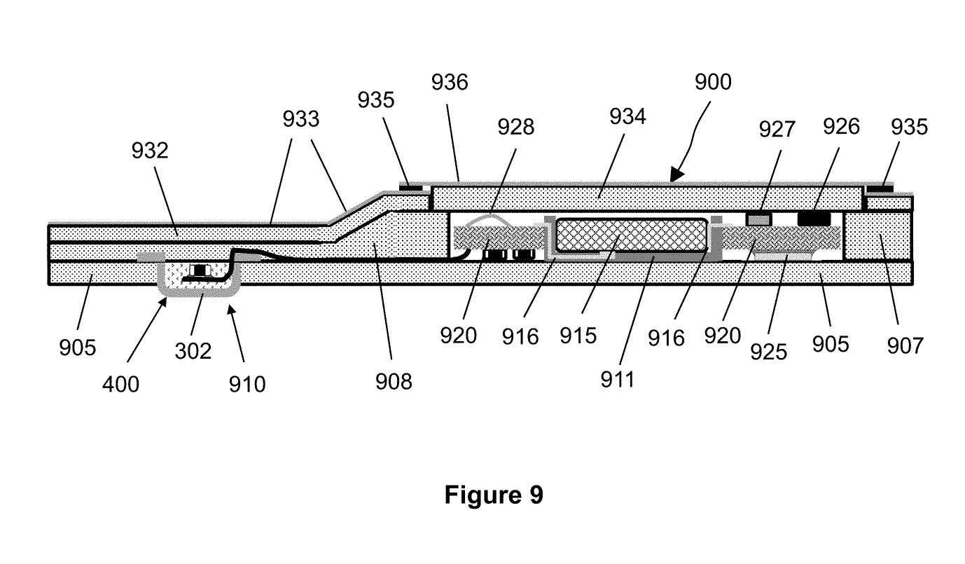

In some embodiments, referring to FIG. 9, a wearable thermometer patch 900 includes a stretchable and permeable layer 905 that include openings 910. The stretchable and permeable layer 905 can be made of soft foam material such as EVA, PE, CR, PORON, EPD, SCF or fabric textile, to provide stretchability and breathability. A temperature probe unit 400, with details described above and shown in FIG. 4, is mounted in the opening 910. A battery holder 911 is soldered into a circuit substrate 920 and attached to an upper surface of the stretchable and permeable layer 905. A replaceable battery 915 can be mounted in the battery holder 911 and electrically connected to connectors 916 to power electronic component such as the temperature probe unit 400. One or more spacers 907 and a wedge-shape spacer 908 are also attached to the upper surface of the stretchable and permeable layer 905 to protect the battery holder 911 and the circuit substrate 920. The one or more spacers 907 and the wedge-shape spacer 908 can be formed by a soft foam material similar to that of the stretchable and permeable layer 905. The wedge-shape spacer 908 defines a varying thickness for the wearable thermometer patch 900. The thicker side of the wedge-shape spacer 908 can be adjacent to the circuit substrate 920 and the battery holder 911 to provide a thicker space. The wedge-shape spacer 908 can advantageously reduce the thickness of the wearable thermometer patch 900 in the unnecessary areas, and can therefore improve flexibility of the wearable thermometer patch 900.

A semiconductor chip 925, an antenna 926, an LED indicator 927, and a switch 928 are mounted on or under the circuit substrate 920. The circuit substrate 920 includes electric circuits therein that electrically connects the various electronic components thereon to the connectors 916 and the replaceable battery 915. The LED indicator 927 can indicate the mode and status (e.g. in measurement mode, off mode, fever warning, etc.) of the wearable thermometer patch 900. The switch 928 is optional and can turn the power from the replaceable battery 915 on or off. In one implementation, the circuit substrate 920 can be implemented with a printed circuit board. The circuit substrate 920 mounted with the various electronic components is bonded to the stretchable and permeable layer 905 by an adhesive layer.

Referring to FIGS. 4 and 9, the temperature probe unit 400, as described above, includes a thermally conductive cup 302 having its bottom portion mounted into the large opening 910 and fixed to the stretchable and permeable layer 905 by an adhesive. A temperature sensor 301 is electrically connected to the electric circuit in the circuit layer 905 by a flexible conductive ribbon 303. The temperature sensor 301 is connected to the electric circuits in the circuit substrate 920 and can send an electric signal to the electric circuit and the semiconductor chip 925 in response to temperature measured by the temperature sensor 301. The semiconductor chip 925 processes the electric signal and outputs another electrical signal which enables the antenna 926 to transmit a wireless signal to send measurement data to an external device such as a mobile phone or a computer. The replaceable battery 915 powers the semiconductor chip 925, the electric circuit, and possibly the temperature sensor 301.

Referring back to FIG. 9, an elastic layer 932 is formed on the one or more spacers 907, and the circuit substrate 920 mounted with various electronic components. A thin film 933 is formed on the elastic layer 450 with an adhesive for protection and cosmetic purposes. In an opening of the elastic layer 932, an elastic cover layer 934 is placed over the battery holder 911 and the replaceable battery 915, and on portions of the one or more spacers 907. The elastic cover layer 934 can be removed to allow removal or replacement of the replaceable battery 915. The elastic cover layer 934 is sealed and held in place by a detachable cover layer 936 with a ring-shaped detachable water resistant adhesive 935.

The elastic layer 932 and the elastic cover layer 934 can be formed by soft stretchable foam and permeable materials such as EVA, PE, CR, PORON, EPD, SCF, or fabric textile. Thus the circuit substrate 920 mounted with the various electronic components, and the replaceable battery 915 are sandwiched between and protected by the stretchable and permeable layer 905, and the elastic layer 932 and the elastic cover layer 934. The spacers 907 provide extra cushion and protection to the temperature probe unit 400 and the above mentioned components.

The semiconductor chip 925 processes the electric signal and outputs an electrical signal which enables the antenna 926 to transmit a wireless signal carrying the measurement data to another external device such as a mobile phone or a computer. The wireless signal can be based on using WiFi, Bluetooth, Near Field Communication (NFC), and other wireless standards. When the wearable thermometer patch 900 is worn by a user, the antenna 926 is separated from the user's skin by the thickness of the circuit substrate 416 and the stretchable and permeable layer 905, which minimizes the impact of the user's body on the transmissions of wireless signals by the antenna 926.

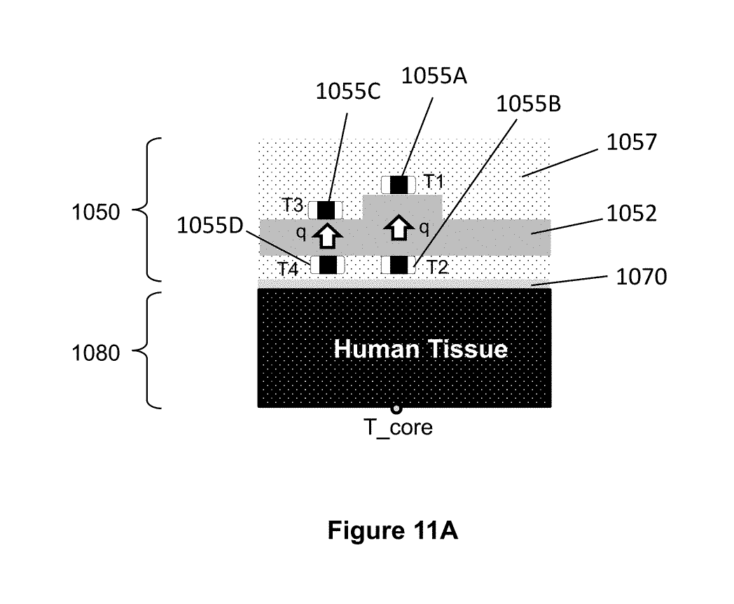

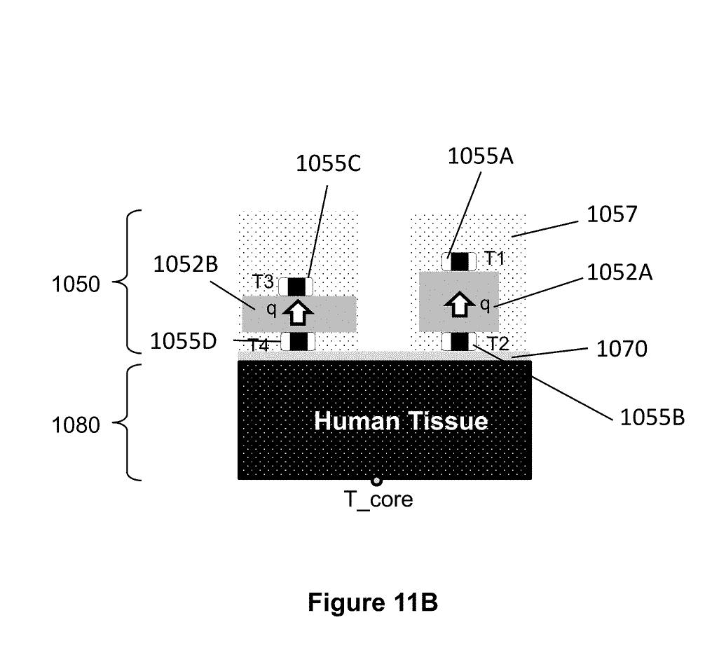

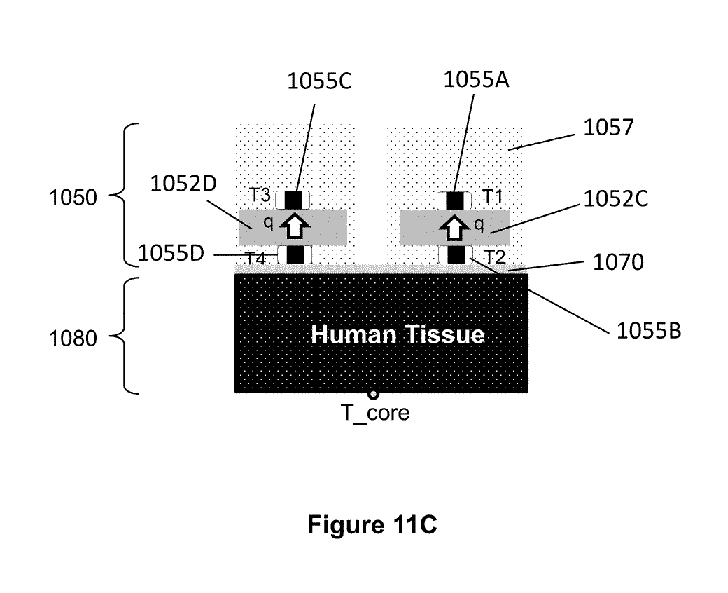

It should be noted that the wearable thermometer patches 800, 900 are compatible with other configurations of temperature probe units. For example, the wearable thermometer patches 800, 900 can respectively incorporate the temperature probe unit 1050 (FIGS. 11A-11C below) by mounting it in an opening of the stretchable and permeable layer 805 or 905. The flexible and conforming wearable thermometer patches 800, 900 are suitable for measuring skin temperature over soft tissues such as under the armpit.

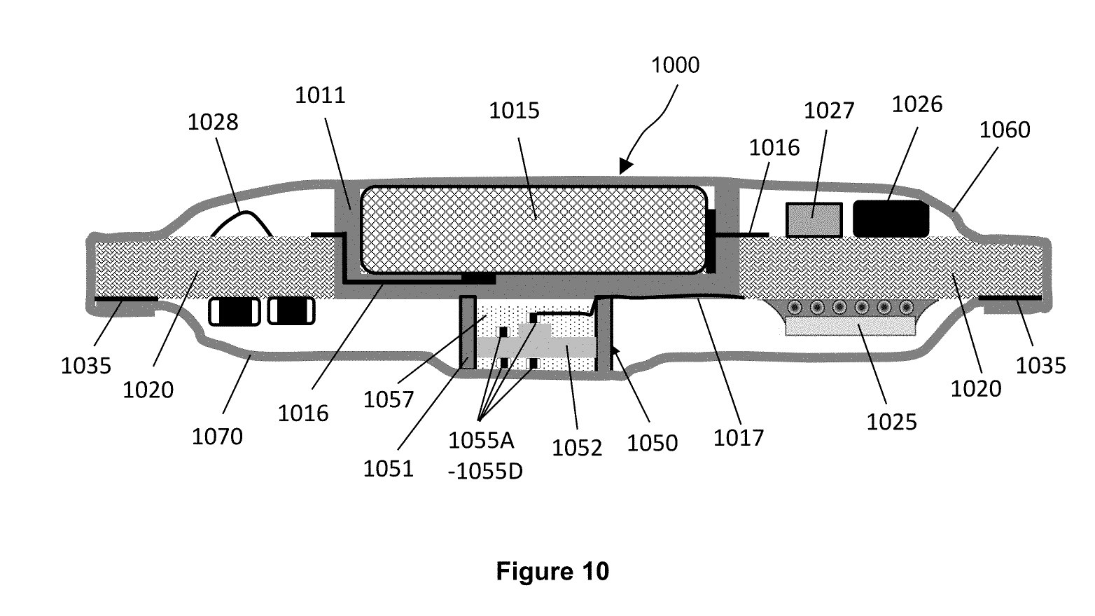

In some embodiments, referring to FIG. 10, a wearable thermometer patch 1000 includes a circuit substrate 1020, a battery holder 1011 is mounted into an opening of the circuit substrate 1020, and a temperature probe unit 1050 attached to an underside of the battery holder 1011 or the circuit substrate 1020. A semiconductor chip 1025, an antenna 1026, an LED indicator 1027, and a switch 1028 are mounted on or under the circuit substrate 1020. A replaceable battery 1015 can be mounted in the battery holder 1011 and electrically connected to connectors 1016 to power electronic component such as the temperature probe unit 1050, the semiconductor chip 1025, the antenna 1026, the LED indicator 1027, and the switch 1028. The circuit substrates 1020 respectively include electric circuits therein that electrically connects the various electronic components thereon to the connectors 1016 and the replaceable battery 1015. The LED indicator 1027 can indicate the mode and status (e.g. in measurement mode, off mode, fever warning, etc.) of the wearable thermometer patch 1000. The switch 1028 is optional and can turn the power from the replaceable battery 1015 on or off. In one implementation, the circuit substrate 1020 can be implemented with a printed circuit board. The circuit substrate 1020 mounted with the various electronic components is bonded to the stretchable and permeable substrate 1005 by an adhesive layer.

A soft detachable cover layer 1060 is attached to the top side top and around the edges of the circuit substrate 1020 and components mounted thereon, the battery holder 1011 and the replaceable battery 1015. The soft detachable cover layer 1060 can be made of an elastic, sticky, and water resistant material such as silicone. The soft detachable cover layer 1060 can be easily detached for the replacement of the replaceable battery 1015. A thermal conductive spreader layer 1070 is attached under the circuit substrate 1020 and the temperature probe unit 1050. The thermal conductive spreader layer 1070 can be bonded to a lower surface of the circuit substrate 1020 by an adhesive 1035 such as Epoxy. Thus, the circuit substrate 1020 and associated electronic components, and the temperature probe unit 1050 are protected from physical abrasion and impact, and moistures, by the soft detachable cover layer 1060 above and the thermal conductive spreader layer 1070 below.

Referring to FIGS. 10 and 11A, the temperature probe unit 1050 includes a housing 1051 that can be implemented with a separate unit or an integrated unit as the battery holder 1011. The temperature probe unit 1050 includes a plate 1052 having a known thermal resistance and temperature sensors 1055A-1055D (respectively measuring temperatures T1-T4) bonded to the top and bottom surfaces of the plate 1052. The plate 1052 can be formed by materials such as plastic, ceramic, metal, or foam materials. The temperature sensors 1055A-1055D can be implemented for example by thermistor, a resistance temperature detector, or thermocouple, which are electrically connected to the circuit in the circuit substrate 1020 via conductive lines 1017. A thermally insulating material 1057 such as Epoxy fills up the housing 1051 and encapsulates the plate 1052 and the temperature sensors 1055A-1055D. When the thermal conductive spreader layer 1070 is in contact with an epidermis and dermis layers 1080 of a human skin, the temperature sensors 1055A-1055D can effectively exchange heat with the human tissue through the thermal conductive spreader layer 1070.

The pair of temperature sensors 1055A, 1055B is respectively attached to the upper surface and the lower surface of the plate 1052 with the temperature sensor 1055A positioned over the temperature sensor 1055B. Similarly, the pair of temperature sensors 1055C, 1055D is respectively attached to the upper surface and the lower surface of the plate 1052 with the temperature sensor 1055C positioned over the temperature sensor 1055D. The plate 1052 has different thicknesses (and thus different thermal resistances) at portions between the temperature sensors 1055A, 1055B and between the temperature sensors 1055C, 1055D.

The temperature sensors 1055A-1055D are electrically connected to the electric circuits in the circuit substrate 1020 and can send an electric signal to the electric circuit and the semiconductor chip 1025 in response to temperature measured by the temperature sensor 301. The semiconductor chip 1025 processes the electric signal and outputs another electrical signal which enables the antenna 1026 to transmit a wireless signal to send measurement data to an external device such as a mobile phone or a computer. The replaceable battery 1015 powers the semiconductor chip 1025, the electric circuit, and possibly the temperature sensors 1055A-1055D.

Referring to FIG. 11B, in another implementation, the temperature probe unit 1050 includes the components and their functions that are similar to what's shown in FIG. 11A and described above except for the plate 1052 (in FIG. 11A) is replaced by two plates 1052A, 1052B separated by a gap in the planar direction. The plates 1052A and 1052B have different thicknesses and are sandwiched respectively between the pair of temperature sensors 1055A, 1055B and between the pair of temperature sensors 1055C, 1055D.

Referring to FIG. 11C, in another implementation, the temperature probe unit 1050 includes the components and their functions that are similar to what's shown in FIG. 11A-11B and described above. The plate 1052 (in FIG. 11A) is replaced by two plates 1052C, 1052D separated by a gap in the planar direction. The plates 1052C and 1052D have different thicknesses and are sandwiched respectively between the pair of temperature sensors 1055A, 1055B and between the pair of temperature sensors 1055C, 1055D.

Referring to FIGS. 10 and 11A-11C, the semiconductor chip 1025 processes the electric signal and outputs an electrical signal which enables the antenna 1026 to transmit a wireless signal carrying the measurement data to another external device such as a mobile phone or a computer. The wireless signal can be based on using WiFi, Bluetooth, Near Field Communication (NFC), and other wireless standards. When the wearable thermometer patch 1000 is worn by a user, the antenna 1026 is separated from the user's skin by the thickness of the circuit substrate 416 and the stretchable and permeable substrate 1005, which minimizes the impact of the user's body on the transmissions of wireless signals by the antenna 1026.

In accordance with the present invention, the assembly of the temperature probe unit 1050 allows accurate measurement of the user's skin temperature. When the diameters of the plates 1052-1052D are large enough compared to their respective thicknesses, the temperature distribution across the surfaces is approximately uniform; one-dimensional Fourier's law can be applied to describe heat conduction in the thickness direction of the plates 1052-1052D (see discussion above in relation to equation (1)). At thermal equilibrium, the heat flux conducted is the same through all the plates and layers in the stack at each planar location. The skin temperature T_core (shown in FIGS. 11A-11C) under the epidermis and dermis layers 1080 can be calculated based on one-dimensional Fourier's law with the following equation (see discussion above in relation to equation (2)):

$${T}_{core} = {T}_{1} + \frac{( {T}_{2} - {T}_{4} ) ( {T}_{2} - {T}_{1} )}{K ( {T}_{4} - {T}_{3} ) - ( {T}_{2} - {T}_{4} )} \qquad eqn . ( 3 )$$

T₁-T₄ are respectively temperatures measured by the temperature sensors 1055A-1055D at the bottom and the top surfaces of the plates 1052-1052D respectively, while K is the ratio of the thermal resistance in the portion of the plate 1052 between T₁ and T₂ (or the plate 1052A in FIG. 11B or 1052C in FIG. 11C) over the thermal resistance in another portion of the plate 1052 between T₃ and T₄ (or the plate 1052B in FIG. 11B or 1052D in FIG. 11C). The calculations of the skin temperature T_core can be conducted by the semiconductor chip 1025 and sent to an external device. The skin temperature T_core can also be calculated by an external device which receives temperature measurement data from the wearable thermometer patch 1000 via wireless communications.

It should be noted that the wearable thermometer patch 1000 is compatible with other configurations of temperature probe units. For example, the wearable thermometer patch 1000 can incorporate the temperature probe unit 400 (FIG. 4) by mounting it in an opening in the circuit substrate 1020.

The flexible and conforming wearable thermometer patch 1000 is suitable for measuring skin temperature over flat skin surface such as.

The disclosed wearable thermometer patches have one or more of the following advantages. The temperature probe unit is integrated with a circuit and a battery holder for holding replaceable battery, which makes the wearable thermometer patches very compact, conforming user's skin, capable of maximum continuous monitoring of user's temperature. The measurement data can also be wirelessly communicated with external devices.

The disclosed wearable thermometer patches can also include electronic components such as the semiconductor chips, resistors, capacitors, inductors, diodes (including for example photo sensitive and light emitting types), other types of sensors, transistors, amplifiers. The sensors can also measure temperature, acceleration and movements, and chemical or biological substances. The electronic components can also include electromechanical actuators, chemical injectors, etc. The semiconductor chips can perform communications, logic, signal or data processing, control, calibration, status report, diagnostics, and other functions.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A wearable thermometer patch for continuous wearing by a user, comprising:
a circuit substrate comprising an electric circuit;
a battery holder mounted in the circuit substrate, wherein the battery holder is configured to hold a replaceable battery to supply power to the electric circuit;
a temperature probe unit in connection with the electric circuit, comprising one or more temperature sensors in electric connection with the electric circuit in the circuit substrate, the one or more temperature sensors each configured to measure temperatures near the user's skin to produce one or more temperature values; and
a detachable cover layer on the battery holder.

2. The wearable thermometer patch of claim 1, further comprising:
a stretchable and permeable layer below the circuit substrate and the battery holder, wherein the temperature probe unit is mounted in an opening of the stretchable and permeable layer, wherein at least a portion of the temperature probe unit is configured to be in contact with the user's skin.

3. The wearable thermometer patch of claim 2, wherein the temperature probe unit includes a thermally conductive cup having a bottom portion configured to be in contact with the user's skin, wherein the one or more temperature sensors are placed inside the thermally conductive cup and are in thermal conduction with the thermally conductive cup.

4. The wearable thermometer patch of claim 2, further comprising:
one or more spacers on the stretchable and permeable layer; and
a thin film on the one or more spacers, wherein the detachable cover is adhesively attached to a portion of the thin film.

5. The wearable thermometer patch of claim 1, wherein the one or more spacers include a wedge-shaped spacer that defines varying thicknesses for the wearable thermometer patch.

6. The wearable thermometer patch of claim 5, wherein the wedge-shaped spacer includes a thinner side and a thicker side, wherein the thicker side is adjacent to the circuit substrate and the battery holder.

7. The wearable thermometer patch of claim 1, further comprising:
an elastic cover layer between the detachable cover layer and the battery holder holding the associated replaceable battery therein.

8. The wearable thermometer patch of claim 1, wherein the temperature probe unit comprises:
a housing;
a first plate in the housing; and
a first pair of temperature sensors in the housing, including:
a first temperature sensor attached to a lower surface of the first plate; and
a second temperature sensor under the first temperature sensor and attached to an upper surface of the first plate.

9. The wearable thermometer patch of claim 8, further comprising:
a thermally insulating material in the housing, which encapsulates the first pair of temperature sensors.

10. The wearable thermometer patch of claim 8, further comprising:
a second pair of temperature sensors in the housing, including:
a third temperature sensor attached to a lower surface of the first plate; and
a fourth temperature sensor under the third temperature sensor and attached to an upper surface of the first plate,
wherein the first plate has a first thickness between the first pair of temperature sensors and a second thickness between the second pair of temperature sensors.

11. The wearable thermometer patch of claim 10, wherein the semiconductor chip is configured to calculate the temperature of the user's skin in part using a difference between temperature values respectively measured by the third temperature sensor and the fourth temperature sensor.

12. The wearable thermometer patch of claim 10, wherein the first thickness is different from the second thickness.

13. The wearable thermometer patch of claim 8, further comprising:
a second plate separated from the first plate by a gap in a planar direction;
a second pair of temperature sensors in the housing, including:
a third temperature sensor attached to a lower surface of a second plate; and
a fourth temperature sensor under the third temperature sensor and attached to an upper surface of the second plate,
wherein the first plate has a first thickness between the first pair of temperature sensors, wherein the second plate has a second thickness between the second pair of temperature sensors.

14. The wearable thermometer patch of claim 13, wherein the semiconductor chip is configured to calculate the temperature of the user's skin in part using a difference between temperature values respectively measured by the third temperature sensor and the fourth temperature sensor.

15. The wearable thermometer patch of claim 13, wherein the first thickness is different from the second thickness.

16. The wearable thermometer patch of claim 13, wherein the first thickness is substantially the same as the second thickness.

17. The wearable thermometer patch of claim 8, further comprising:
a semiconductor chip mounted on the circuit substrate and in electric connection with the electric circuit, wherein the semiconductor chip is configured to receive electric signals from the one or more temperature sensors in response to respective temperatures measured from the user's skin.

18. The wearable thermometer patch of claim 17, wherein the semiconductor chip is configured to calculate the temperature of the user's skin in part using a difference between temperature values respectively measured by the first temperature sensor and the second temperature sensor.

19. The wearable thermometer patch of claim 8, further comprising:
a thermal conductive spreader layer attached below the housing of the temperature probe unit and the circuit substrate.

20. The wearable thermometer patch of claim 1, further comprising:
a semiconductor chip mounted on the circuit substrate and in electric connection with the electric circuit; and
an antenna in electric connection with the semiconductor chip, wherein the antenna is configured to wirelessly send temperatures measured by the one or more temperature sensors or calculated temperature values to an external device.
